(19) United States
(12) Patent Application Publication
(10) Pub. No.: US 2023/0201117 A1
(43) Pub. Date: Jun. 29, 2023

(54) OPHTHALMIC NANOEMULSION COMPOSITIONS

(71) Applicant: LUPIN LIMITED, Maharastra Mumbai (IN)

(72) Inventors: Hemant Hanumant BHALERAO, Maharashtra Pune (IN); Sajeev CHANDRAN, Maharashtra Pune (IN)

(21) Appl. No.: 17/999,924

(22) PCT Filed: May 26, 2021
(86) PCT No.: PCT/IB2021/054560

§ 371 Date: Nov. 27, 2022

(30) Foreign Application Priority Data
May 27, 2020 (IN) 202021022222

(51) Int. Cl. A61K 9/107; A61K 31/542; A61K 31/5575; A61K 31/498; A61K 9/00; A61K 47/44; A61K 47/26; A61K 47/10; A61K 47/36

(52) U.S. Cl. CPC A61K 9/1075; A61K 31/542; A61K 31/5575; A61K 31/498; A61K 9/0048; A61K 47/44; A61K 47/26; A61K 47/10; A61K 47/36

(57) ABSTRACT

The present invention relates to sterile pharmaceutical nanoemulsion ophthalmic composition comprising Brinzolamide, Brimonidine and Bimatoprost useful in the treatment of ocular complications such as glaucoma. The nanoemulsion composition of the invention may also comprise in situ gelling composition with or without preservative(s). The invention also relates to processes for making such compositions.

### FIELD OF THE INVENTION

The present invention relates to sterile pharmaceutical nanoemulsion ophthalmic composition comprising Brinzolamide, Brimonidine and Bimatoprost useful in the treatment of ocular complications such as glaucoma. The nanoemulsion composition of the invention may also comprise in situ gelling composition with or without preservative(s). The invention also relates to processes for making such compositions.

### BACKGROUND OF THE INVENTION

Worldwide, Glaucoma is one of the leading ocular causes of blindness, affecting more than about 50 million populations. Glaucoma constitutes a disease that may run undetected for several years resulting in considerable damage to retinal ganglion cells and nerve fiber layer of retina. Glaucoma has generally been classified into open-angle and closed-angle, primary and secondary, and further classified into acute and chronic forms. Elevated intraocular pressure (TOP) constitutes a major risk factor for glaucoma. (Tham, Yih-Chung, et al Ophthalmology 121.11 (2014): 2081-2090; Adams, Christopher M., et al. Pharmaceutical research 36.2 (2019): 25).

Research in the field of ophthalmology aiming to the treatment of glaucoma suggest use of different classes of drugs, collectively designed to lower TOP by either decreasing aqueous humour production rate or increasing aqueous humour outflow or a combination of both mechanisms.

Conventional ophthalmic drops of prostaglandin analogues, beta-blockers, alpha-adrenergic agonists, carbonic anhydrase inhibitors, miotics (cholinergic agonists) and adrenergic agonists such as epinephrine and its derivatives and their combinations are available for treatment of glaucoma. Several single active as well as combination ophthalmic products are available in the market. Some of these include Azopt® suspension (brinzolamide suspension); Symbrinza® suspension (Brimonidine and Brinzolamide); Alphagan® solution (brimonidine tartarate); Combigan® solution (Brimonidine, Timolol); Lumigan® solution (bimatoprost) Kritantek® solution (Brimonidine, Dorzolamide and Timolol); Tripligan® Solution (Brimonidine, Timolol and Bimatoprost).

US 2018/0169092 A1 discloses method for treating or mitigating glaucoma by using pharmaceutical composition comprising combination of first component, second component, third component, and fourth component. It discloses formulations such as solutions or as colloidal systems (i.e., suspensions or emulsions). US 2019/0008920 A1 discloses ophthalmic composition comprising at least two active pharmaceutical ingredients. It discloses nanoemulsion composition. US 2017/0304316 discloses ophthalmic compositions comprising carbonic anhydrase inhibitors(s) alone or in combination of other intraocular pressure (TOP) reducing active ingredients. US2018/0353504 A1 discloses a low dose brimonidine ophthalmic composition along with second glaucoma drug, an antihistamine or NSAID. Bhalerao et al discloses in situ gelling nanoemulsion formulations of brinzolamide. (Bhalerao, et al. Drug delivery and translational research 10.2 (2020): 529-547.)

While some of these currently available treatments may provide lowering of intraocular pressure, very few of these have been proved to cure or even arrest the progression of glaucoma. Several treatments have been associated with undesired side effects such as burning, stinging sensations, blurred vision, dry eye and the limitations of requirement of multiple dosing (3 to 4 times per day) and dosing inaccuracy. Therefore there is an unmet need in the art for providing better pharmacologic interventions which can demonstrate substantial lowering of intraocular pressure.

It was observed that most of the available ophthalmic products comprise either solution or suspension dosage forms. It is well known in the art that; the bioavailability of the suspension formulation is reduced because of the fact that the drug must be dissolved in order to be absorbed before being eliminated from the eye surface. The suspension formulation also suffer from the drawback that the particulates have a potential to cause irritation, increasing the likelihood that they would be rubbed or washed from the eye. Additionally, manufacturing of sterile suspension formulation for ophthalmic use has its own challenges like particle size reduction, degree of homogenization required and ability of the formulation to remains in to the suspended state without particle aggregation throughout the shelf life.

Other types of vehicles are known for the purpose of increasing residence time of the drug on the eye surface. Hydrogels or ophthalmic ointments are known to increase the bioavailability by means of an increase of the viscosity of the composition; however the success in the increase of bioavailability was very limited. Further the ophthalmic ointments have large inconvenience of the awkwardness of their application and blurred vision being produced after their application. Likewise, a large number of novel vehicles have been reported such as liposomes, nanoparticles, etc., though most of them have problems of stability, tolerance, difficulties for industrialization and limited success as far as the increase of bioavailability is concerned.

The present invention relates to sterile pharmaceutical nanoemulsion ophthalmic compositions of the multiple therapeutic actives, which can deliver the actives in therapeutic dosage to the eye with the convenience of dosing of any other mono-active ophthalmic solution. The composition according to the invention offers an advantage of synergistic effects of the combination of actives in a single stable formulation thereby providing different molecular targets for the pathogenesis of ocular implications such as Glaucoma. The composition according to the invention demonstrates improved bioavailability, pharmacokinetic profile and pharmaceutical performance.

Brimonidine is an exemplary active belonging to the class of “α-adrenergic agonists” refers to a class of sympathomimetic agents that selectively stimulates α-adrenergic receptors (i.e., a group of proteins that sense molecules outside the cell and activate inside signal transduction pathways and cellular responses). It inhibits the activity of adenyl cyclase enzyme, reducing the activation of sympathetic nervous system mediated by the motor vessel centre of the spinal cord. In this way, it has two mechanisms of action (a) decreases the production of aqueous humor and (b) increases the outflow through the uveo-scleral route. The active ingredient Brimonidine as used herein may be a free base or any pharmaceutically acceptable salt such as tartarate and the like, or derivative thereof.

Brinzolamide is an exemplary active belonging to the class of carbonic anhydrase inhibitors. It inhibits the enzyme which catalyzes the dissociation of carbonic acid in to H₂O and CO₂. In this way, it reduces the production of aqueous humor. The active ingredient Brinzolamide as used herein may be a free base or any pharmaceutically acceptable salt or derivative thereof

Bimatoprost is an exemplary active belonging to the class of prostaglandin analogues. It works by increasing the outflow of aqueous humor, opening the trabecular meshwork and relaxing the ciliary muscle. The active ingredient Bimatoprost as used herein may be a free base or any pharmaceutically acceptable salt or derivative thereof.

The current invention offers an advantage of synergistic effects of the combination of Brinzolamide, Brimonidine and Bimatoprost in a single stable ophthalmic formulation thereby providing different molecular targets for the pathogenesis of ocular implications such as Glaucoma. The composition according to the invention demonstrates improved bioavailability, pharmacokinetic profile and pharmaceutical performance.

### SUMMARY OF THE INVENTION

The inventors of the present invention have developed pharmaceutical nanoemulsion ophthalmic compositions comprising Brinzolamide, Brimonidine and Bimatoprost which can deliver the multiple active agents in therapeutic dosage to the eye with the convenience of dosing of any other mono-active ophthalmic solution. The current invention offers a combination formulation consisting of Brinzolamide, Brimonidine and Bimatoprost providing different molecular targets for the pathogenesis of ocular implications such as Glaucoma. Specific combination of the actives with different mechanism of action in single formulation provides regulations of more than one molecular pathway and provides a significant benefit to Glaucoma patients. The invention also relates to processes for making such compositions and use of said compositions in patient populations including pediatric populations.

In one of the embodiments of the invention, the inventors of the present invention have developed the in-situ gelling nanoemulsion ophthalmic compositions of brinzolamide, brimonidine, bimatoprost which can deliver drug at the right dosage to the eye with the convenience of dosing just like any other ophthalmic solution. Entrapment of one or more of the actives in oil phase of nanoemulsion can avoid the degradation, metabolism and cellular efflux during the course of drug delivery. Application method of in-situ gelling nanoemulsion is as simple as that of any conventional drops, while avoids any discomfort associated with the other dosage forms such as inserts or implants.

In an embodiment of the present invention, there is provided a pharmaceutical nanoemulsion ophthalmic composition comprising Brinzolamide, Brimonidine and Bimatoprost; about 0.2% to about 25% w/v of oil phase dispersed in an aqueous phase; a primary and/or a secondary surfactant and optionally a thickener; wherein at least one of the Brinzolamide, Brimonidine, Bimatoprost are present in the oil phase and wherein the nanoemulsion composition exhibits a mean globule size not more than 200 nm.

In another embodiment of the invention, there is provided a pharmaceutical in-situ gelling nanoemulsion ophthalmic compositions comprising Brinzolamide, Brimonidine and Bimatoprost; about 0.2% to about 25% w/v of oil phase dispersed in an aqueous phase; a primary and/or a secondary surfactant; a thickener, and optionally one or more preservative; wherein at least one of the Brinzolamide, Brimonidine, Bimatoprost are present in the oil phase; and wherein the nanoemulsion composition exhibits a mean globule size not more than 200 nm.

In yet another embodiment of the invention there is provided a stable pharmaceutical in-situ gelling nanoemulsion ophthalmic compositions comprising Brinzolamide, Brimonidine and Bimatoprost; about 0.2% to about 25% w/v of oil phase dispersed in an aqueous phase; a primary and/or a secondary surfactant; a thickener, and optionally one or more preservative; wherein at least one of the Brinzolamide, Brimonidine, Bimatoprost are present in the oil phase; and wherein the nanoemulsion composition exhibits a mean globule size not more than 200 nm.

In another embodiment of the present invention there is provided a pharmaceutical in-situ gelling nanoemulsion ophthalmic compositions comprising of oil in water type emulsion that increases the bioavailability of the actives. Said emulsion is stable during storage without the need of including in its composition the potentially irritating products and ones that can cause cataractogenic process.

In yet another embodiment of the invention there is provided a pharmaceutical in-situ gelling nanoemulsion ophthalmic compositions comprising Brinzolamide, Brimonidine and Bimatoprost; about 0.2% to about 25% w/v of oil phase dispersed in an aqueous phase; a primary and/or a secondary surfactant; a thickener, and optionally one or more preservative; wherein at least one of the Brinzolamide, Brimonidine, Bimatoprost are present in the oil phase; and wherein the nanoemulsion composition exhibits improved pharmacokinetic profile.

In another embodiment of the invention there is provided a pharmaceutical in-situ gelling nanoemulsion ophthalmic compositions comprising Brinzolamide, Brimonidine and Bimatoprost; about 0.2% to about 25% w/v of oil phase dispersed in an aqueous phase; a primary and/or a secondary surfactant; a thickener, and optionally one or more preservative; wherein at least one of the Brinzolamide, Brimonidine, Bimatoprost are present in the oil phase; and wherein the nanoemulsion composition exhibits in vitro gelling time of not more than about 45 seconds.

In another embodiment of the invention there is provided preservative free pharmaceutical nanoemulsion ophthalmic compositions comprising Brinzolamide, Brimonidine and Bimatoprost; about 0.2% to about 25% w/v of oil phase dispersed in an aqueous phase; a primary and/or a secondary surfactant; a thickener, and wherein the composition is stored in specially designed container closure.

In another embodiment of the invention there is provided preservative free pharmaceutical in-situ gelling nanoemulsion ophthalmic compositions comprising Brinzolamide, Brimonidine and Bimatoprost; about 0.2% to about 25% w/v of oil phase dispersed in an aqueous phase; a primary and/or a secondary surfactant; a thickener, and wherein the composition is stored in specially designed container closure.

In another embodiment of the invention there is provided a pharmaceutical nanoemulsion ophthalmic compositions comprising Low dose Brinzolamide, Brimonidine and Bimatoprost; about 0.2% to about 25% w/v of oil phase dispersed in an aqueous phase; a primary and/or a secondary surfactant and optionally a thickener; wherein at least one of the Brinzolamide, Brimonidine, Bimatoprost are present in the oil phase and wherein the nanoemulsion composition exhibits a mean globule size not more than 200 nm.

In yet another embodiment of the invention there is provided a pharmaceutical in situ gelling nanoemulsion ophthalmic compositions comprising Low dose Brinzolamide, Brimonidine and Bimatoprost; about 0.2% to about 25% w/v of oil phase dispersed in an aqueous phase; a primary and/or a secondary surfactant and a thickener; wherein at least one of the Brinzolamide, Brimonidine, Bimatoprost are present in the oil phase and wherein the nanoemulsion composition exhibits a mean globule size not more than 200 nm. In yet another embodiment, there is provided a pharmaceutical nanoemulsion ophthalmic composition to be obtained with normal emulsification equipment, with a rotary agitator or a pressurized homogenizer. In another embodiment there is provided a pharmaceutical in-situ gelling nanoemulsion ophthalmic composition to be obtained with normal emulsification equipment, with a rotary agitator or a pressurized homogenizer.

### DETAILED DESCRIPTION OF THE INVENTION

As used herein, “or” means “and/or” unless stated otherwise. Furthermore, use of the term “including” as well as other forms, such as “includes,” and “included,” is not limiting.

As used herein, “About” means that a member referred to as “about” comprises the recited number plus or minus 1-10% of that recited member. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3. etc., up to and including only 20.

As used herein, the terms “active(s)”, “active agent”, “active ingredient” and “active pharmaceutical ingredient” are used interchangeably herein and refer to a compound that is used or known to one skilled in the art in treating an eye disorder, such as glaucoma. The active ingredient(s) as used herein may be a free base or any pharmaceutically acceptable salt or derivative thereof.

As used herein, the term pharmaceutically acceptable salt encompasses a salt formed from pharmaceutically acceptable non-toxic acids including inorganic or organic acids. There is no limitation regarding the salts, except that if used for therapeutic purposes, they must be pharmaceutically acceptable. Salts of the active ingredient(s) may be prepared from pharmaceutically acceptable non-toxic acids, including inorganic and organic acids. Such acids include among others acetic, benzene sulfonic, benzoic, camphor sulfonic, citric, ethansulfonic, fumaric, gluconic, glutamic, hydrobromic, hydrochloric, lactic, maleic, malic, mandelic, methanesulfonic, phosphoric, succinic, sulphuric, tartaric, and p-toluensulfonic acid. In one of the embodiment of the invention, the active ingredient Brimonidine as used herein may be a free base or tartarate salt of brimonidine.

As used herein, the term pharmaceutically acceptable ester encompasses an ester formed from pharmaceutically acceptable non-toxic acids including inorganic or organic acids. There is no limitation regarding the ester, except that if used for therapeutic purposes, they must be pharmaceutically acceptable. Non limiting examples of such acids include among others acetic, butyric, propionic, benzenesulfonic, benzoic, camphorsulfonic, citric, ethansulfonic, fumaric, gluconic, glutamic, hydrobromic, hydrochloric, lactic, maleic, malic, mandelic, methanesulphonic, phosphoric, succinic, sulphuric, tartaric and p-toluensulphonic acid.

Although the present invention is described with respect to the composition of Brinzolamide, Brimonidine and Bimatoprost, the invention is not limited thereto. The composition according to the invention may also include additional active agents, non-limiting examples of which include beta-adrenergic receptor agonist(s); immunosuppressant(s); lymphocyte associated antigen antagonist(s); anti-inflammatory agent(s); beta-blocker (s); alpha 2 adrenergic receptor agonist(s); prostaglandin analog(s); carbonic anhydrase inhibitor(s); histamine receptor antagonist(s); anti-infective(s), antibiotic(s), corticosteroid(s), antihistaminic/mast cell stabilisers or combinations thereof.

Non limiting examples of corticosteroid(s) may include prednisolone, methylprednisolone, difluprednate, prednisone acetate, prednisolone, sodium phosphate, triamcinolone, fluocinolone; fluorometholone, betamethasone, medrysone, rimexolone, dexamethasone, hydrocortisone, loteprednol and a combination thereof. Non limiting examples of anti-inflammatory agent(s) may include non-steroidal anti-inflammatory drug (“NSAID”), thymosin beta 4, and a combination thereof. In one particular instances, the NSAID is selected from the group consisting of diclofenac, flubiprofen, ketorolac, 5 ketorolac thromethamine, bromfenac, nepafenac, flurbiprofen, and a combination thereof. Non limiting examples of beta-adrenergic receptor agonist(s) may include dopexamine, epinephrine, isoprenaline, isoproterenol, levalbuterol, salbutamol, albuterol, and a combination thereof. Non limiting examples of beta blocker(s) may include timolol, propranolo, sotalol, nadolol, betaxolol, levobetaxolol and a combination thereof. Non limiting examples of prostaglandins analog(s) may also include latanoprost, travoprost, tafluprost, and a combination thereof. Additional non limiting examples of carbonic anhydrase inhibitor(s) is selected from the group consisting of dorzolamide, methazolamide, dichlorphenamide, and a combination thereof. Additional non limiting examples of α-2 adrenergic receptor agonist(s) may include 4-NEMD, 7-Me-marsanidine, agmatine, apraclonidine, cannabigerol, clonidine, detomidine, dexmedetomidine, fadolmidine, guanabenz, guanfacine, lofexidine, marsanidine, medetomidine, methamphetamine, mivazerol, rilmenidine, romifidine, talipexole, tizanidine, tolonidine, xylazine, xylometazoline, and the like. Non limiting examples of antibiotics and/or antibacterial may include gatifloxacin, besifloxacin, neomycin; polymyxin tobramycin, sulfacetamide sodium, gentamicin, oxytetracycline, natamycin, chloramphenicol, tetracycline and a combination thereof. Non limiting example of antihistamine/mast cell stabiliser include, but are not limited to, levocabastine, alcaftadine, azelastine, bepotastine, emedastine, epinastine, ketotifen, olopatadine and a combination thereof.

The present invention relates to pharmaceutical nanoemulsion ophthalmic composition comprising Brinzolamide, Brimonidine and Bimatoprost useful in the prophylaxis and/or treatment of ocular implications such as glaucoma. The nanoemulsion composition of the invention may also comprise in situ gelling composition with or without preservative(s). The invention also relates to processes for making such compositions and use of said compositions in patient populations including pediatric populations.

In one of the embodiment of the invention, the composition includes from about 0.001% to about 1% w/v of brinzolamide or a salt thereof. In another embodiment, the amount of brinzolamide present in the composition is about 0.75% w/v.

In another of the embodiment of the invention, the composition includes from about 0.001% to about 1% w/v of brimonidine or a salt thereof (e.g., brimonidine tartrate and hydroxy brimonidine trifluoroacetate). In another embodiment, the amount of brimonidine tartrate present in the composition is about 0.15% w/v.

In another embodiment of the invention, the composition includes from about 0.001% to about 1% w/v of bimatoprost or a salt thereof. In one embodiment, the amount of bimatoprost present in the composition is about 0.02% w/v.

As used herein, the term “nanoemulsion” refers to oil in water emulsion having a mean emulsion droplet particle size OR mean globule size Dx (50)) of not more than about 200 nm, typically not more than about 150 nm, often not more than about 100 nm, and most often not more than about 80 nm. In one of the preferred embodiments the mean emulsion droplet particle size of the nanoemulsion is in the range of about 10 nm to about 100 nm. In accordance with the present invention the globule size were measured by using Delsa™ Nano C, Beckman Coulter connected with Delsa™ Nano UI software version 3.73.

In accordance with the present invention “In-situ gelling nanoemulsion” is an emulsion that undergoes gelation in situ (at physiological site of application), to form a gel within not more than 45 seconds of administration. In one of the embodiments the emulsion forms gel within 30 seconds of administration.

Wherein the viscosity of gel formed in-situ is at least twice of the initial formulation. In one of the preferred embodiment, viscosity of gel formed in-situ is increased at least 3 to 9 times of the initial formulation. In most preferred embodiment viscosity of gel formed in-situ is increased at least 5-7 times of initial formulation. In accordance with the invention, the viscosity of the nanoemulsion was determined by viscometer (Brookfield Engineering Labs Inc) at 25.0±0.5° C. The rotation speed was 100 rpm and the viscosity was determined in centipoise from rheogram plotted using shear stress (dyn/cm²) and shear rate (1/s). In one of the embodiments of the present invention, the viscosity of the composition may be between about 50 cps to about 500 cps. In one of the preferred embodiment of the present invention viscosity of the composition may be between about 100 cps to about 400 cps. In another preferred embodiment of the invention, the viscosity of the composition is between about 150 cps to about 300 cps.

The in situ gelling nanoemulsion compositions according to the invention provide a wide variety of surprising unexpected benefits including, but not limited to, extended release of the actives, better penetration profile of the active pharmaceutical ingredient(s) to the desired cells, increased resident time in the cornea, etc.

In accordance with the present invention, In situ gelling time was determined by placing a drop of the nanoemulsion composition of the present invention in a vial containing about 2 mL of pH 7.4 stimulated tear fluid (STF).

In accordance with the present invention, the nanoemulsion has a pH in the range of about 5 and about 8 and an osmolality between about 250 and about 800 mOsm/kg. The appearance of these emulsions tends to be light milky.

In accordance with the present invention, the oil soluble or partly oil soluble drugs such as brinzolamide, brimonidine, bimatoprost are included in an oil in water type emulsion to be administered in the eye thus increasing the bioavailability of the actives. Said vehicle comprises an oil and a non-ionic surfactant as well as optionally one or more preservative to meet the requirements of the pharmacopeia.

The oil that forms part of the emulsion may be one or more of vegetable oil, animal oil, mineral oil, fatty acids, medium chain triglyceride, fatty alcohol or any combination of these oils and/or oily substances that are well tolerated at the eye level. Non limiting examples of the oils are medium chain triglycerides, vegetable oil, olive oil, sunflower seed oil, sesame seed oil with an acid value less than 0.5, castor oil, mineral oil and glyceryl 5 monostearate (GMS) and/or combination thereof. In preferred embodiments, the oils are castor oil, mineral oil and glyceryl monostearate (GMS) or combination thereof. In one of the aspect of the invention, the oil phase is selected from the group consisting of vegetable oil(s), animal oil(s), mineral oil(s), fatty acid(s), medium chain triglyceride(s), fatty alcohol(s) or combination thereof.

In accordance with the present invention the oil phase contains at least one of the Brinzolamide, Brimonidine and/or Bimatoprost completely or partially solubilized in the oil phase. In one of the embodiments of the invention, the nanoemulsion composition includes Brinzolamide:Brimonidine:Bimatoprost present in an amount of about 0.25:0.05:0.005% (w/v) respectively. In another embodiment of the invention, the nanoemulsion composition includes Brinzolamide:Brimonidine:Bimatoprost present in an amount of about 0.5:0.1:0.01% (w/v) respectively. In yet another embodiment of the invention, the nanoemulsion composition includes Brinzolamide:Brimonidine:Bimatoprost present in an amount of about 0.75:0.15:0.015% (w/v) respectively or 0.75:0.15:0.02% (w/v) respectively or 1:0.2:0.03% respectively.

In accordance with the present invention, oil is present in concentration preferably between about 0.2% to about 25% w/v of composition. In a preferred embodiment the oil phase is present between about 5% w/v to about 25% w/v of composition. In most preferred embodiment oil component is present in about 5% w/v to about 12.5% w/v of composition.

In accordance with the present invention the aqueous phase includes water or water and glycerin mixture. In one of the embodiment the aqueous phase may also include hydroalcoholic mixtures.

In accordance with the present invention, the surfactant may be primary or secondary surfactant. In accordance of present invention “primary surfactant” is a surfactant used in to increase solubility of actives in oil. In accordance of present invention “secondary surfactant” is used as emulsifier in aqueous phase of emulsion. Non limiting examples of primary surfactant and secondary surfactant may include, but not limited to, sorbitan esters (such as Span or Arlacel), glycerol esters (such as glycerin monostearate), polyethylene glycol esters (such as polyethylene glycol stearate), block polymers (such as poloxamers (Pluronics®)), acrylic polymers (such as Pemulen®), ethoxylated fatty esters (such as polyoxyl 35 castor oil, Cremophor® RH-40), ethoxylated alcohols (such as Brij®), ethoxylated fatty 30 acids (such as polysorbate 80, Tween or Tween 20), monoglycerides, silicon based surfactants alone or in combination. In a preferred embodiment, the primary surfactant and secondary surfactant is polyoxyl 35 castor oil and polysorbate 80 used alone or in combination. In one of the preferred embodiment primary surfactant is polyoxyl 35 castor oil and secondary surfactant is polysorbate 80. In one of the embodiment of the invention the nanoemulsion composition includes primary surfactant in the range of about 2.5 to about 20% w/v of the composition and secondary surfactant in the range of about 2.5 to about 20% w/v of the composition. In more preferred embodiments the primary surfactant polyoxyl 35 castor oil is present in an amount of about 5 to about 15% w/v of the total composition. In another preferred embodiment the primary surfactant polyoxyl 35 castor oil is present in an amount of about 7.5% w/v of the total composition. In another preferred embodiment the secondary surfactant polysorbate 80 is present in an amount of about 5 to about 15% w/v of the total composition. In another preferred embodiment the primary surfactant polysorbate 80 is present in an amount of about 5% w/v of the total composition.

In preferred embodiment of the present invention the ratio of primary surfactant to secondary surfactant is 1:2, 1:1.5, 1:1, 1.5:1, 2:1, 2.5:1, 3:1 or fractions in between. In more preferred embodiment the ratio of primary to secondary surfactant is 1.5:1.

In accordance with the present invention, the thickener is a polymer material having an ion-sensitive characteristics. In accordance with the present invention Ionic strength dependent in-situ gel polymers are polymers that undergo a sol-gel transition upon contact with physiological ions (Na⁺, K⁺, Ca⁺⁺, Mg⁺⁺ etc.) in the site of application or site of action. In accordance with the invention, in-situ gel polymer may be used alone or in combination. Non limiting examples of in-situ gel polymers may include various types of sodium alginate, gellan gum, guar gum, pectin and sodium hyaluronate. In one of the embodiment of the invention, the thickener is gellan gum. In another embodiment of the invention, in-situ gel polymer can be combined with other thickening polymers such as one or more of hypromellose (HPMC), methyl cellulose (MC), polyvinyl pyrrolidone (PVP), polyvinylalcohol (PVA), and Poly(acrylic acid) polymers such as carbomers, and the like.

In accordance with the present invention, thickener may be present in concentration preferably about 0.10% w/v to about 0.50% w/v of the composition. In a preferred embodiment thickener is present in about 0.15% to about 0.30% w/v of the composition. In most preferred embodiment thickener is present in about 0.15% to about 0.25% w/v of the composition. In one of the most preferred embodiments the present invention comprises gellan gum as a thickener in concentration about 0.20% to 0.25% w/v of the composition.

In accordance with the present invention, the composition may optionally further comprise of one or more of the following components; isotonizing agents, stabilizers, buffers, preservatives and/or antioxidants.

The composition of the present invention may include an isotonizing agent such as mannitol, glycerin, glycerol, sorbitol, glucose or combination thereof. In preferred embodiment the isotonizing agent is glycerin, glycerol or combination thereof. The composition of present invention may contain stabilizers such as sodium edatate, citric acid or combination thereof; buffers such as tris(hydroxymethyl)aminomethane (tromethamine), sodium phosphate and potassium phosphate, sodium citrate, sodium carbonate and sodium bicarbonate or combination thereof; preservatives such as quaternary ammonium compound like benzalkonium chloride, chlorobutanol, sodium perborate or combination thereof. In a preferred embodiment the preservative used is benzalkonium chloride.

In one of the embodiment of the invention, there is provided preservative free pharmaceutical in-situ gelling nanoemulsion ophthalmic compositions comprising Brinzolamide, Brimonidine and Bimatoprost; about 0.2% to about 25% w/v of oil phase dispersed in an aqueous phase; a primary and/or a secondary surfactant; a thickener, and wherein the composition is stored in specially designed container closure which prevent microbial ingress into the container and retains the sterility of the content during its shelf life and in-use period. Non limiting example of such specially designed container closure is NOVELIA® manufactured by Nemera

As used herein, the term “preservative” for the purposes of the present invention refers to a chemical substance that is added to a pharmaceutical composition to prevent the pharmaceutical composition from deterioration, decomposition or degradation or to substantially reduce or decelerate the degree and/or the speed of such deterioration, decomposition or degradation.

As used herein, the term “preservative-free” means a pharmaceutical composition that does not include preservative.

The compositions of the present invention may be sterilized by filtration or they may be obtained by sterilization of the aqueous phase and the oily phase and subsequently mixing and emulsifying in aseptic conditions.

It will be readily apparent to one skilled in the art that varying substitutions and modifications may be made to the invention disclosed herein without departing from the scope and spirit of the invention. For example, those skilled in the art will recognize that the invention may be practiced using a variety of different compounds within the described generic descriptions.

### Examples

Tables I (A, B, C); II (A, B, C) and III (A, B, C) depicts the exemplary examples illustrating qualitative and quantitative composition of nanoemulsion and in situ gelling nanoemulsion compositions according to the invention. The examples should not be considered as limiting the scope of this invention in any way, as these examples and other equivalents thereof will become apparent to those versed in the art, in the light of the present disclosure, and the claims.

TABLE I
Examples of Nanoemulsion formulations
(A)

| | | Example-1 | | Example-2 | | Example-3 | | Example-4 |
|---|---|---|---|---|---|---|---|---|
| | | Prototype | Prototype | Prototype | Prototype | Prototype | Prototype | Prototype |
| Sr. | | formula | formula | formula | formula | formula | formula | formula |
| No | Ingredients | (mg/mL) | % w/v | (mg/mL) | % w/v | (mg/mL) | % w/v | (mg/mL) |

A) Oil Phase (Drug Solution Phase)

| | | | | | | | | |
|---|---|---|---|---|---|---|---|---|
| 1 | Brinzolamide | 2.50 | 0.25 | 5.00 | 0.50 | 7.50 | 0.75 | 7.50 |
| 2 | Castor Oil | 50.00 | 5.00 | 50.00 | 5.00 | 50.00 | 5.00 | 8.00 |
| 3 | Polyoxyl 35 | 75.00 | 7.50 | 75.00 | 7.50 | 75.00 | 7.50 | 150 |
| | Castor Oil | | | | | | | |
| 4 | Polysorbate 80 | — | — | — | — | — | — | — |

B) Aqueous Phase

| | | | | | | | | |
|---|---|---|---|---|---|---|---|---|
| 5 | Brimonidine | 0.50 | 0.05 | 1.00 | 0.10 | 1.50 | 0.15 | 1.50 |
| | Tartrate | | | | | | | |
| 6 | Bimatoprost | 0.05 | 0.005 | 0.10 | 0.01 | 0.15 | 0.015 | 0.2 |
| 7 | Polysorbate 80 | 50.00 | 5.00 | 50.00 | 5.00 | 50.00 | 5.00 | 150 |
| 8 | Tromethamine | 1.50 | 0.15 | 1.50 | 0.15 | 1.50 | 0.15 | 1.50 |
| 9 | Sodium borate | — | — | — | — | — | — | — |
| 10 | Boric acid | — | — | — | — | — | — | — |
| 11 | Sodium | — | — | — | — | — | — | — |
| | hydroxide (1M) | | | | | | | |
| 12 | Benzalkonium | 0.50 | 0.05 | 0.50 | 0.05 | 0.50 | 0.05 | — |
| | Chloride | | | | | | | |
| 13 | Glycerin | 50.00 | 5.00 | 50.00 | 5.00 | 50.00 | 5.00 | — |
| 14 | Orthophosphoric | Q.S. | Q.S. | Q.S. | Q.S. | Q.S. | Q.S. | Q.S. |
| | acid (1M) | | | | | | | |
| 15 | Purified Water | Q.S. to 1 mL | Q.S. | Q.S. to 1 mL | Q.S. | Q.S. to 1 mL | Q.S. | Q.S. to 1 mL |

(A)

| | Example-4 | Example-5 | | Example-6 | | Example-7 | |
|---|---|---|---|---|---|---|---|
| | Prototype | Prototype | Prototype | Prototype | Prototype | Prototype | Prototype |
| Sr. | formula | formula | formula | formula | formula | formula | formula |
| No | % w/v | (mg/mL) | % w/v | (mg/mL) | % w/v | (mg/mL) | % w/v |

A) Oil Phase (Drug Solution Phase)

| | | | | | | | |
|---|---|---|---|---|---|---|---|
| 1 | 0.75 | 7.50 | 0.75 | 7.50 | 0.75 | 7.50 | 0.75 |
| 2 | 0.80 | 8.00 | 0.80 | 8.00 | 0.80 | 8.00 | 0.80 |
| 3 | 15 | 150 | 15 | 150 | 15 | 150 | 15 |
| 4 | — | — | — | 150 | 15 | 150 | 15 |

B) Aqueous Phase

| | | | | | | | |
|---|---|---|---|---|---|---|---|
| 5 | 0.15 | 1.50 | 0.15 | 1.50 | 0.15 | 1.50 | 0.15 |
| 6 | 0.02 | 0.2 | 0.02 | 0.2 | 0.02 | 0.2 | 0.02 |
| 7 | 15 | 150 | 15 | — | — | — | — |
| 8 | 0.15 | — | — | 1.50 | 0.15 | — | — |
| 9 | — | 0.1 | 0.01 | — | — | 0.1 | 0.01 |
| 10 | — | 1 | 0.1 | — | — | 1 | 0.1 |
| 11 | — | Q.S. | Q.S. | — | — | Q.S. | Q.S. |
| 12 | — | — | — | — | — | — | — |
| 13 | — | — | — | — | — | — | — |
| 14 | Q.S. | — | — | Q.S. | Q.S. | — | — |
| 15 | Q.S. | Q.S. to 1 mL | Q.S. | Q.S. to 1 mL | Q.S. | Q.S. to 1 mL | Q.S. |

(B)

| | | Example-8 | | Example-9 | | Example-10 | |
|---|---|---|---|---|---|---|---|
| | | Prototype | Prototype | Prototype | Prototype | Prototype | Prototype |
| Sr. | | formula | formula | formula | formula | formula | formula |
| No | Ingredients | (mg/mL) | % w/v | (mg/mL) | % w/v | (mg/mL) | % w/v |

A) Oil Phase (Drug Solution Phase)

| | | | | | | | |
|---|---|---|---|---|---|---|---|
| 1 | Brinzolamide | 2.50 | 0.25 | 5.00 | 0.50 | 7.50 | 0.75 |
| 2 | Brimonidine Tartrate | 0.50 | 0.05 | 1.00 | 0.10 | 1.50 | 0.15 |
| 3 | Castor Oil | 50.00 | 5.00 | 50.00 | 5.00 | 50.00 | 5.00 |
| 4 | Polyoxyl 35 Castor Oil | 75.00 | 7.50 | 75.00 | 7.50 | 75.00 | 7.50 |

B) Aqueous Phase

| | | | | | | | |
|---|---|---|---|---|---|---|---|
| 5 | Bimatoprost | 0.05 | 0.005 | 0.10 | 0.01 | 0.15 | 0.015 |
| 6 | Polysorbate 80 | 50.00 | 5.00 | 50.00 | 5.00 | 50.00 | 5.00 |
| 7 | Tromethamine | 1.50 | 0.15 | 1.50 | 0.15 | 1.50 | 0.15 |
| 8 | Benzalkonium Chloride | 0.50 | 0.05 | 0.50 | 0.05 | 0.50 | 0.05 |
| 9 | Glycerin | 50.00 | 5.00 | 50.00 | 5.00 | 50.00 | 5.00 |
| 10 | Orthophosphoric acid (1M) | Q.S. | Q.S. | Q.S. | Q.S. | Q.S. | Q.S. |
| 11 | Purified Water | Q.S. to 1 mL | Q.S. | Q.S. to 1 mL | Q.S. | Q.S. to 1 mL | Q.S. |

(C)

| | | Example-11 | | Example-12 | | Example-13 | |
|---|---|---|---|---|---|---|---|
| | | Prototype | Prototype | Prototype | Prototype | Prototype | Prototype |
| Sr. | | formula | formula | formula | formula | formula | formula |
| No | Ingredients | (mg/mL) | % w/v | (mg/mL) | % w/v | (mg/mL) | % w/v |
| A) Oil Phase (Drug Solution Phase) | | | | | | | |
| 1 | Brinzolamide | 2.50 | 0.25 | 5.00 | 0.50 | 7.50 | 0.75 |
| 2 | Brimonidine Tartrate | 0.50 | 0.05 | 1.00 | 0.10 | 1.50 | 0.15 |
| 3 | Bimatoprost | 0.05 | 0.005 | 0.10 | 0.01 | 0.15 | 0.015 |
| 4 | Castor Oil | 50.00 | 5.00 | 50.00 | 5.00 | 50.00 | 5.00 |
| 5 | Polyoxyl 35 Castor Oil | 75.00 | 7.50 | 75.00 | 7.50 | 75.00 | 7.50 |
| B) Aqueous Phase | | | | | | | |
| 6 | Polysorbate 80 | 50.00 | 5.00 | 50.00 | 5.00 | 50.00 | 5.00 |
| 7 | Tromethamine | 1.50 | 0.15 | 1.50 | 0.15 | 1.50 | 0.15 |
| 8 | Benzalkonium Chloride | 0.50 | 0.05 | 0.50 | 0.05 | 0.50 | 0.05 |
| 9 | Glycerin | 50.00 | 5.00 | 50.00 | 5.00 | 50.00 | 5.00 |
| 10 | Orthophosphoric acid (1M) | Q.S. | Q.S. | Q.S. | Q.S. | Q.S. | Q.S. |
| 11 | Purified Water | Q.S. to 1 mL | Q.S. | Q.S. to 1 mL | Q.S. | Q.S. to 1 mL | Q.S. |

TABLE-II
Examples of In-situ Gelling Nanoemulsion formulations
(A)

| | | Example-14 | | Example-15 | | Example-16 | |
|---|---|---|---|---|---|---|---|
| | | Prototype | Prototype | Prototype | Prototype | Prototype | Prototype |
| Sr. | | formula | formula | formula | formula | formula | formula |
| No | Ingredients | (mg/mL) | % w/v | (mg/mL) | % w/v | (mg/mL) | % w/v |

A) Oil Phase (Drug Solution Phase)

| | | | | | | | |
|---|---|---|---|---|---|---|---|
| 1 | Brinzolamide | 2.50 | 0.25 | 5.00 | 0.50 | 7.50 | 0.75 |
| 2 | Castor Oil | 50.00 | 5.00 | 50.00 | 5.00 | 50.00 | 5.00 |
| 3 | Polyoxyl 35 Castor Oil | 75.00 | 7.50 | 75.00 | 7.50 | 75.00 | 7.50 |

B) Aqueous Phase

| | | | | | | | |
|---|---|---|---|---|---|---|---|
| 4 | Brimonidine Tartrate | 0.50 | 0.05 | 1.00 | 0.10 | 1.50 | 0.15 |
| 5 | Bimatoprost | 0.05 | 0.005 | 0.10 | 0.01 | 0.15 | 0.015 |
| 6 | Polysorbate 80 | 50.00 | 5.00 | 50.00 | 5.00 | 50.00 | 5.00 |
| 7 | Tromethamine | 1.50 | 0.15 | 1.50 | 0.15 | 1.50 | 0.15 |
| 8 | Gellan Gum | 2.00 | 0.20 | 2.00 | 0.20 | 2.00 | 0.20 |
| 9 | Benzalkonium Chloride | 0.50 | 0.05 | 0.50 | 0.05 | 0.50 | 0.05 |
| 10 | Glycerin | 50.00 | 5.00 | 50.00 | 5.00 | 50.00 | 5.00 |
| 11 | Orthophosphoric acid (1M) | Q.S. | Q.S. | Q.S. | Q.S. | Q.S. | Q.S. |
| 12 | Purified Water | Q.S. to 1 mL | Q.S. | Q.S. to 1 mL | Q.S. | Q.S. to 1 mL | Q.S. |

(B)

| | | Example-17 | | Example-18 | | Example-19 | |
|---|---|---|---|---|---|---|---|
| | | Prototype | Prototype | Prototype | Prototype | Prototype | Prototype |
| Sr. | | formula | formula | formula | formula | formula | formula |
| No | Ingredients | (mg/mL) | % w/v | (mg/mL) | % w/v | (mg/mL) | % w/v |

A) Oil Phase (Drug Solution Phase)

| | | | | | | | |
|---|---|---|---|---|---|---|---|
| 1 | Brinzolamide | 2.50 | 0.25 | 5.00 | 0.50 | 7.50 | 0.75 |
| 2 | Brimonidine Tartrate | 0.50 | 0.05 | 1.00 | 0.10 | 1.50 | 0.15 |
| 3 | Castor Oil | 50.00 | 5.00 | 50.00 | 5.00 | 50.00 | 5.00 |
| 4 | Polyoxyl 35 Castor Oil | 75.00 | 7.50 | 75.00 | 7.50 | 75.00 | 7.50 |

B) Aqueous Phase

| | | | | | | | |
|---|---|---|---|---|---|---|---|
| 5 | Bimatoprost | 0.05 | 0.005 | 0.10 | 0.01 | 0.15 | 0.015 |
| 6 | Polysorbate 80 | 50.00 | 5.00 | 50.00 | 5.00 | 50.00 | 5.00 |
| 7 | Tromethamine | 1.50 | 0.15 | 1.50 | 0.15 | 1.50 | 0.15 |
| 8 | Gellan Gum | 2.00 | 0.20 | 2.00 | 0.20 | 2.00 | 0.20 |
| 9 | Benzalkonium Chloride | 0.50 | 0.05 | 0.50 | 0.05 | 0.50 | 0.05 |
| 10 | Glycerin | 50.00 | 5.00 | 50.00 | 5.00 | 50.00 | 5.00 |
| 11 | Orthophosphoric acid (1M) | Q.S. | Q.S. | Q.S. | Q.S. | Q.S. | Q.S. |
| 12 | Purified Water | Q.S. to 1 mL | Q.S. | Q.S. to 1 mL | Q.S. | Q.S. to 1 mL | Q.S. |

(C)

| | | Example-20 | | Example-21 | | Example-22 | |
|---|---|---|---|---|---|---|---|
| | | Prototype | Prototype | Prototype | Prototype | Prototype | Prototype |
| Sr. | | formula | formula | formula | formula | formula | formula |
| No | Ingredients | (mg/mL) | % w/v | (mg/mL) | % w/v | (mg/mL) | % w/v |

A) Oil Phase (Drug Solution Phase)

| | | | | | | | |
|---|---|---|---|---|---|---|---|
| 1 | Brinzolamide | 2.50 | 0.25 | 5.00 | 0.50 | 7.50 | 0.75 |
| 2 | Brimonidine Tartrate | 0.50 | 0.05 | 1.00 | 0.10 | 1.50 | 0.15 |
| 3 | Bimatoprost | 0.05 | 0.005 | 0.10 | 0.01 | 0.15 | 0.015 |
| 4 | Castor Oil | 50.00 | 5.00 | 50.00 | 5.00 | 50.00 | 5.00 |
| 5 | Polyoxyl 35 Castor Oil | 75.00 | 7.50 | 75.00 | 7.50 | 75.00 | 7.50 |

B) Aqueous Phase

| | | | | | | | |
|---|---|---|---|---|---|---|---|
| 6 | Polysorbate 80 | 50.00 | 5.00 | 50.00 | 5.00 | 50.00 | 5.00 |
| 7 | Tromethamine | 1.50 | 0.15 | 1.50 | 0.15 | 1.50 | 0.15 |
| 8 | Gellan Gum | 2.00 | 0.20 | 2.00 | 0.20 | 2.00 | 0.20 |
| 9 | Benzalkonium Chloride | 0.50 | 0.05 | 0.50 | 0.05 | 0.50 | 0.05 |
| 10 | Glycerin | 50.00 | 5.00 | 50.00 | 5.00 | 50.00 | 5.00 |
| 11 | Orthophosphoric acid (1M) | Q.S. | Q.S. | Q.S. | Q.S. | Q.S. | Q.S. |
| 12 | Purified Water | Q.S. to 1 mL | Q.S. | Q.S. to 1 mL | Q.S. | Q.S. to 1 mL | Q.S. |

TABLE III
Examples of Preservative Free In-Situ Gelling Nanoemulsion formulations
(A)

| | | Example-23 | | Example-24 | | Example-25 | | Example- 26 | |
|---|---|---|---|---|---|---|---|---|---|
| | | Prototype | Prototype | Prototype | Prototype | Prototype | Prototype | Prototype | Prototype |
| Sr. | | formula | formula | formula | formula | formula | formula | formula | formula |
| No | Ingredients | (mg/mL) | % w/v | (mg/mL) | % w/v | (mg/mL) | % w/v | (mg/mL) | % w/v |

A) Oil Phase (Drug Solution Phase)

| | | | | | | | | | |
|---|---|---|---|---|---|---|---|---|---|
| 1 | Brinzolamide | 2.50 | 0.25 | 5.00 | 0.50 | 7.50 | 0.75 | 7.5 | 0.75 |
| 2 | Castor Oil | 50.00 | 5.00 | 50.00 | 5.00 | 50.00 | 5.00 | 8.0 | 0.80 |
| 3 | Polyoxyl 35 | 75.00 | 7.50 | 75.00 | 7.50 | 75.00 | 7.50 | 150 | 15 |
| | Castor Oil | | | | | | | | |

B) Aqueous Phase

| | | | | | | | | | |
|---|---|---|---|---|---|---|---|---|---|
| 4 | Brimonidine | 0.50 | 0.05 | 1.00 | 0.10 | 1.50 | 0.15 | 1.5 | 0.15 |
| | Tartrate | | | | | | | | |
| 5 | Bimatoprost | 0.05 | 0.005 | 0.10 | 0.01 | 0.15 | 0.015 | 0.2 | 0.02 |
| 6 | Polysorbate 80 | 50.00 | 5.00 | 50.00 | 5.00 | 50.00 | 5.00 | 150 | 15 |
| 7 | Tromethamine | 1.50 | 0.15 | 1.50 | 0.15 | 1.50 | 0.15 | 1.5 | 0.15 |
| 8 | Gellan Gum | 2.00 | 0.20 | 2.00 | 0.20 | 2.00 | 0.20 | 1.5 | 0.15 |
| 9 | Glycerin | 50.00 | 5.00 | 50.00 | 5.00 | 50.00 | 5.00 | — | — |
| 10 | Orthophosphoric | Q.S. | Q.S. | Q.S. | Q.S. | Q.S. | Q.S. | Q.S. | Q.S. |
| | acid (1M) | | | | | | | | |
| 11 | Purified Water | Q.S. to 1 mL | Q.S. | Q.S. to 1 mL | Q.S. | Q.S. to 1 mL | Q.S. | Q.S. to 1 mL | Q.S. to 1 mL |

(B)

| | | Example-27 | | Example-28 | | Example-29 | |
|---|---|---|---|---|---|---|---|
| | | Prototype | Prototype | Prototype | Prototype | Prototype | Prototype |
| Sr. | | formula | formula | formula | formula | formula | formula |
| No | Ingredients | (mg/mL) | % w/v | (mg/mL) | % w/v | (mg/mL) | % w/v |

A) Oil Phase (Drug Solution Phase)

| | | | | | | | |
|---|---|---|---|---|---|---|---|
| 1 | Brinzolamide | 2.50 | 0.25 | 5.00 | 0.50 | 7.50 | 0.75 |
| 2 | Brimonidine Tartrate | 0.50 | 0.05 | 1.00 | 0.10 | 1.50 | 0.15 |
| 3 | Castor Oil | 50.00 | 5.00 | 50.00 | 5.00 | 50.00 | 5.00 |
| 4 | Polyoxyl 35 Castor Oil | 75.00 | 7.50 | 75.00 | 7.50 | 75.00 | 7.50 |

B) Aqueous Phase

| | | | | | | | |
|---|---|---|---|---|---|---|---|
| 5 | Bimatoprost | 0.05 | 0.005 | 0.10 | 0.01 | 0.15 | 0.015 |
| 6 | Polysorbate 80 | 50.00 | 5.00 | 50.00 | 5.00 | 50.00 | 5.00 |
| 7 | Tromethamine | 1.50 | 0.15 | 1.50 | 0.15 | 1.50 | 0.15 |
| 8 | Gellan Gum | 2.00 | 0.20 | 2.00 | 0.20 | 2.00 | 0.20 |
| 9 | Glycerin | 50.00 | 5.00 | 50.00 | 5.00 | 50.00 | 5.00 |
| 10 | Orthophosphoric acid (1M) | Q.S. | Q.S. | Q.S. | Q.S. | Q.S. | Q.S. |
| 11 | Purified Water | Q.S. to 1 mL | Q.S. | Q.S. to 1 mL | Q.S. | Q.S. to 1 mL | Q.S. |

(C)

| | | Example-30 | | Example-31 | | Example-32 | |
|---|---|---|---|---|---|---|---|
| | | Prototype | Prototype | Prototype | Prototype | Prototype | Prototype |
| Sr. | | formula | formula | formula | formula | formula | formula |
| No | Ingredients | (mg/mL) | % w/v | (mg/mL) | % w/v | (mg/mL) | % w/v |

A) Oil Phase (Drug Solution Phase)

| | | | | | | | |
|---|---|---|---|---|---|---|---|
| 1 | Brinzolamide | 2.50 | 0.25 | 5.00 | 0.50 | 7.50 | 0.75 |
| 2 | Brimonidine Tartrate | 0.50 | 0.05 | 1.00 | 0.10 | 1.50 | 0.15 |
| 3 | Bimatoprost | 0.05 | 0.005 | 0.10 | 0.01 | 0.15 | 0.015 |
| 4 | Castor Oil | 50.00 | 5.00 | 50.00 | 5.00 | 50.00 | 5.00 |
| 5 | Polyoxyl 35 Castor Oil | 75.00 | 7.50 | 75.00 | 7.50 | 75.00 | 7.50 |

B) Aqueous Phase

| | | | | | | | |
|---|---|---|---|---|---|---|---|
| 6 | Polysorbate 80 | 50.00 | 5.00 | 50.00 | 5.00 | 50.00 | 5.00 |
| 7 | Tromethamine | 1.50 | 0.15 | 1.50 | 0.15 | 1.50 | 0.15 |
| 8 | Gellan Gum | 2.00 | 0.20 | 2.00 | 0.20 | 2.00 | 0.20 |
| 9 | Glycerin | 50.00 | 5.00 | 50.00 | 5.00 | 50.00 | 5.00 |
| 10 | Orthophosphoric acid (1M) | Q.S. | Q.S. | Q.S. | Q.S. | Q.S. | Q.S. |
| 11 | Purified Water | Q.S. to 1 mL | Q.S. | Q.S. to 1 mL | Q.S. | Q.S. to 1 mL | Q.S. |

### Manufacturing Process:

The nanoemulsion compositions were prepared by using combination of high speed and high pressure homogenization technique.

1. The drug solution phase (oil phase) was prepared by dissolving either one or more of Brinzolamide, Brimonidine tartrate, Bimatoprost in mixture of castor oil and polyoxyl 35 castor oil preheated at 50-80° C. under stirring using magnetic stirrer. Optionally polysorbate 80 may be added to it.

2. In aqueous phase; tris (hydroxymethyl) aminomethane (Tris buffer) was added in purified water and its pH was adjusted in between 6.8 to 7.2 using phosphoric acid.

3. The aqueous phase was then heated at 40-50° C. along with the actives, polysorbate 80, and optionally gellan gum (in case of in situ gelling nanoemulsion composition), glycerol and benzalkonium chloride (not in case of preservative free in situ gelling nanoemulsion) were added under stirring.

4. Drug solution phase was added drop wise in aqueous phase under homogenization at 2000 to 3000 RPM and homogenization continued for 45-60 minutes.

5. This micro emulsion then passed through high pressure homogenizer at 1000-1200 bar pressure for 30 minutes.

6. Prepared nanoemulsion filtered through 1.0-micron filter to remove particulate matter present in nanoemulsion.

7 Sterilization of above nanoemulsion was performed using filtration techniques using 0.45 micron followed by 0.22-micron filter.

The compositions produced were subjected to physico-chemical characterizations in terms of determination of drug content (Assay), pH, globule size, viscosity, osmolality, specific gravity, and degradation profiling. The compositions were observed to be clear to milky white in appearance with a pH of about 7.0. The viscosity of the formulations was found to be 225-232 cps. Further the osmolality of the formulations was found to be in the range of 559 to 662 mOsm/kg while specific gravity of the formulations was found in the range of 1.000 to 1.050. Results of physico-chemical characterizations including the drug content (Assay) and degradation profiles for the Examples 4, 7 and 26 are listed in Table IV.

TABLE IV
Physicochemical characterization

| | Example 4 | Example 7 | Example 26 |
|---|---|---|---|
| pH | 7.0 | 7.0 | 7.05 |
| Mean globule | 12 | 15 | 15 |
| size (nm) | | | |

Assay (% w/v)

| | | | |
|---|---|---|---|
| Brinzolamide | 99.7 | 100.3 | 100.0 |
| Brimonidine | 100.6 | 102.3 | 95.4 |
| Tartrate | | | |
| Bimatoprost | 102.4 | 95.2 | 103.6 |

Degradation Products (%)
A) Brimonidine Tartrate degradation profile

| | | | |
|---|---|---|---|
| Impurity A | Not detected | Not detected | Not detected |
| Impurity B | Not detected | 0.10 | 0.08 |
| Impurity C | Not detected | Not detected | Not detected |
| Impurity D | 0.19 | Not detected | 0.19 |
| Impurity E | Below detection | Below detection | Not detected |
| | limit | limit | |
| Impurity F | 0.06 | Not detected | 0.01 |
| Impurity G | Not detected | Not detected | 0.02 |
| Impurity H | Not detected | Not detected | Not detected |
| Impurity I | Not detected | Not detected | Not detected |
| 4-DMP | Not detected | Not detected | Not detected |
| Highest unknown | 0.26 | 0.0 | 0.21 |
| impurity | | | |
| Total degradation | 0.77 | 0.10 | 0.31 |
| products | | | |

B) Brinzolamide degradation profile

| | | | |
|---|---|---|---|
| Impurity E | 0.04 | 0.04 | 0.04 |
| Impurity B | 0.21 | Not detected | 0.15 |
| Impurity C | Not detected | Below detection | Not detected |
| | | limit | |
| Impurity D | 0.11 | Below detection | 0.15 |
| | | limit | |
| Highest unknown | 0.06 | 0 | 0.09 |
| impurity | | | |
| Total degradation | 0.53 | 0.04 | 0.73 |
| products | | | |

C) Bimatoprost degradation profile

| | | | |
|---|---|---|---|
| Bimatoprost acid | 0.0 | Not detected | 0.1 |
| 15-epi bimatoprost | Not detected | Not detected | Not detected |
| 5,6-trans | Not detected | Not detected | Not detected |
| bimatoprost | | | |
| Highest unknown | 0.36 | 0.07 | 0.0 |
| impurity | | | |
| Total degradation | 1.17 | 0.07 | 0.10 |
| products | | | |

The stability studies of selected compositions were carried out as per International Conference on Harmonization guidelines after storage of the formulations for 6 months. The storage conditions employed were ambient (25° C.±2° C./40±5% RH) and accelerated (40° C.±2° C./25±5% RH) stability condition. The required volume of in-situ gelling nanoemulsion was stored in closed LDPE bottles and sealed tightly. At predetermined time intervals, samples were withdrawn and studied for the characteristics such as description, drug content, particle size, Zeta potential, pH, osmolality, water loss and in vitro drug release profile. The in-vivo pharmacodynamic studies were carried out using glaucomatous rabbits. The reference product and the test composition according to the invention are administered at same dose. TOP measurement carried out by using calibrated Schiotz tonometer at different time.

1. A pharmaceutical nanoemulsion ophthalmic compositions comprising Brinzolamide, Brimonidine and Bimatoprost;
about 0.2% to about 25% w/v of oil phase dispersed in an aqueous phase;
a primary and/or a secondary surfactant;
optionally a thickener;
optionally one or more preservative;
wherein at least one of the Brinzolamide, Brimonidine, Bimatoprost are present in the oil phase.

2. The nanoemulsion composition according to claim 1, wherein oil phase is selected from the group consisting of vegetable oil(s), animal oil(s), mineral oil(s), fatty acid(s), medium chain triglyceride(s), fatty alcohol(s) or combination thereof

3. The nanoemulsion composition according to claim 1, wherein primary surfactant and secondary surfactant are selected from the group consisting of sorbitan esters, glycerol esters, polyethylene glycol esters, block polymers, acrylic polymers, ethoxylated fatty esters, ethoxylated alcohols, ethoxylated fatty acids, monoglycerides, silicon based 20 surfactants or combination thereof.

4. The nanoemulsion composition according to claim 1, wherein the nanoemulsion composition exhibits a mean globule size not more than 200 nm.

5. A pharmaceutical in-situ gelling nanoemulsion ophthalmic compositions comprising Brinzolamide, Brimonidine and Bimatoprost;
about 0.2% to about 25% w/v of oil phase dispersed in an aqueous phase;
a primary and/or a secondary surfactant;
a thickener, and
optionally one or more preservative;
wherein at least one of the Brinzolamide, Brimonidine, Bimatoprost are present in the oil phase.

6. The nanoemulsion composition according to claim 5, wherein oil phase is selected from the group consisting of vegetable oil(s), animal oil(s), mineral oil(s), fatty acid(s), medium chain triglyceride(s), fatty alcohol(s) or combination thereof

7. The nanoemulsion composition according to claim 5, wherein primary surfactant and secondary surfactant are selected from the group consisting of sorbitan esters, glycerol esters, polyethylene glycol esters, block polymers, acrylic polymers, ethoxylated fatty esters, ethoxylated alcohols, ethoxylated fatty acids, monoglycerides, silicon based 20 surfactants or combination thereof.

8. The nanoemulsion composition according to claim 5, wherein thickener comprises a polymeric material having an ion-sensitive characteristics.

9. The nanoemulsion according to claim 5, wherein the thickener is selected from the group consisting of, sodium alginate, gellan gum, guar gum, pectin, sodium hyaluronate, hydroxypropyl methylcellulose, methyl cellulose, polyvinyl pyrrolidone, polyvinylalcohol, poly (acrylic acid) polymers or combination thereof.

10. The nanoemulsion composition according to claim 5, wherein the nanoemulsion composition exhibits a mean globule size not more than 200 nm.
